(19) United States
(12) Patent Application Publication
(10) Pub. No.: US 2026/0115408 A1
(43) Pub. Date: Apr. 30, 2026

(54) BRAIN PHOTO-STIMULATION DEVICE IN THE FORM OF A STROBOSCOPIC LIGHT DEVICE

(71) Applicant: DREAM MACHINE TECHNOLOGY, Paris (FR)

(72) Inventors: Brice BATTUNG, Strasbourg (FR); Kevin FINEL, L’hay-les-Roses (FR)

(21) Appl. No.: 19/116,037

(22) PCT Filed: Sep. 27, 2023
(86) PCT No.: PCT/EP2023/076782

§ 371 Date: Mar. 27, 2025

(30) Foreign Application Priority Data
Sep. 29, 2022 (FR) 2209930

(51) Int. Cl. A61M 21/00; H05B 45/32

(52) U.S. Cl. CPC A61M 21/00; H05B 45/32; A61M 2021/0044

(57) ABSTRACT

The brain photo-stimulation device includes flashing light sources and a mechanism for managing the flashing frequency of the sources in a housing. The mechanism for managing the flashing frequency of the light sources includes at least two oscillators. Each oscillator controls at least the flashing frequency of at least one group. Each group includes at least one flashing light source and a controller for a respective oscillator. The at least one light source generates its own strobe frequency, which, together with at least one other strobe frequency, forms a light signal in the direction of a user.

### CROSS-REFERENCE TO RELATED APPLICATIONS

See Application Data Sheet.

### STATEMENT REGARDING FEDERALLY SPONSORED RESEARCH OR DEVELOPMENT

Not applicable.

### THE NAMES OF PARTIES TO A JOINT RESEARCH AGREEMENT

Not applicable.

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not applicable.

### STATEMENT REGARDING PRIOR DISCLOSURES BY THE INVENTOR OR A JOINT INVENTOR

Not applicable.

### BACKGROUND OF THE INVENTION

### 1. Field of the Invention

The present invention relates to the field of photo-stimulation devices.

The main purpose of the present invention is to propose a photo-stimulation device, in particular a brain photo-stimulation device, in the form of a stroboscopic light device which, by means of light, acts on a user's cognition, resulting, in particular, in an altered state of consciousness and the creation of an introspective state.

### 2. Description of Related Art Including Information Disclosed Under 37 CFR 1.97 And 37 CFR 1.98

The photo-stimulation device generally comprises a source emitting light perceived by the user's eye, and a control device for controlling said source.

Traditionally, in the state of the art light therapy devices have been used to expose the user to an artificial light source with an intensity and light spectrum close to that of sunlight.

Such devices are generally used to regulate circadian rhythm disorders, seasonal depression or classic depression.

However, such devices do not allow the user's state of consciousness to be influenced.

Also known, in the state of the art, from the international application published under number WO 2010/139480, is a light therapy device comprising, compulsorily, on the one hand, a continuous, or constant, uninterrupted light source whose intensity and color can vary and, on the other hand, a flickering light source that is superimposed on the first continuous light source so that the user is subjected to both light sources simultaneously.

The frequency of the flickering light is increased by a control module, between the start of the experience, from an initial frequency, and the end of the experience, where the aim is to reach a target frequency that is at least twice the initial frequency, to induce a psychophysical experience in the subject by accelerating the flicker frequency.

Thus, in this document, it is only intended that the user experience the phenomenon of flicker acceleration during a session.

Such acceleration, induced by an increase in the flicker frequency of light sources between the start and end of the session, can have harmful effects on some users.

Additionally, as the light therapy device necessarily requires at least one continuous light source and a plurality of flickering light sources, it is a complicated design.

Also known from the state of the art is US patent document US2010/130812, which describes a device for modulating projected light so as to generate modulated color component output signals for a light projection device.

The device described herein comprises a light modulator which generates color component (red, green and blue) output signals according to both an intensity and color parameter, the color parameter, and, in communication with said modulator, a color modulation generator for generating the modulated color signal.

This device, whose purpose is to create a soft, flickering vibration with an imperceptible effect, does not generate a stroboscopic signal, via a flashing light phenomenon, perceived by the user.

Additionally, since this device requires means of modulating signals of different colors, it also has a complicated design.

Also known from US patent document US2021/023332 is a system for promoting, in particular, a user's sleep, which system receives, from at least one sensor, data associated with one or more biometric markers of the user in order to determine a brain state, based on the data associated with the biometric marker(s), before determining a desired altered cognitive state of the user and causing a transmitter device to apply a stimulus to the user.

### BRIEF SUMMARY OF THE INVENTION

The aim of such a system is to facilitate the user's circadian training, and in particular to promote sleep.

In this system, the light-emitting device comprises a first light and a second light, the first light being configured to emit a first wavelength of light, the second light being configured to emit a second wavelength.

The lights are controlled by a controller, based on data collected by biometric information sensors, including heart rate, blood flow, blood pressure, body temperature and so on. Based on this data, the controller system determines or predicts the user's cognitive state and determines the stimulus to be applied to the user to modify it. In addition to being visual, the stimulus applied can be auditory, tactile, etc. . . .

Here, the system, based on the presence of a plurality of biometric sensors designed to collect information on the user's state, also has a complex design.

The present invention is intended to remedy, at least in part, the disadvantages of the devices known from the state of the art.

In an inventive approach, a brain photo-stimulation device has been devised comprising only LED (light-emitting diode) type flashing light sources, arranged in a plurality of independently controllable groups, so as to generate different strobe frequencies which accumulate to form a single stroboscopic signal, inducing a gentle modification of the user's brain activity, through stimulation, initially, of the latter's optic nerve.

To this end, the invention relates to a photo-stimulation device in the form of a stroboscopic light device, comprising at least, in a housing, a plurality of flashing light sources in the form of LEDs and means for managing the flashing frequency of said light sources.

Said photo-stimulation device according to the present invention is particular in that said means for managing the flashing frequency of the light sources comprise, on the one hand, at least two oscillators, each of the oscillators controlling the flashing frequency, the light intensity and the on/off duty cycle of at least one group comprising at least one flashing light source and, on the other hand, means for controlling said oscillators, so that each group comprising at least one light source generates its own strobe frequency, the addition of which with at least one other strobe frequency forms a stroboscopic light signal in the direction of a user.

Additionally, according to a particular feature of the stroboscopic device of the invention, said control means control said oscillators based on a computer file in the form of a code of a stroboscopic light session to be projected to the user, the session being made up of at least two successive steps, each step being defined by an index, a duration d and, at the beginning and end of the step, the flashing frequency f, the duty cycle R of a pulse-width-modulated square-wave signal applied to the flashing frequency (or duty cycle) and the luminous intensity L of the LEDs of the LED group(s) to be controlled, the values f, R and L being modified and recalculated at a given periodicity of between 80 ms and 120 ms.

According to particular embodiments of the present photo-stimulation device:
said control means of said oscillators consist of a microcontroller;
it comprises four oscillators;
it comprises nine groups, each with at least one and at most three flashing light source(s), the latter consisting of LEDs.

The present invention also relates to a method for managing sequences of control signals from the oscillators to the drivers of the groups of LEDs in a brain photo-stimulation device according to the invention.

Such a method is characterized by the generation of at least one sequence of control signals by the oscillators, based on a program selected from a plurality of predefined programs stored in the control means, the sequence consisting of at least two successive steps, each step performing a frequency and intensity calculation for LED control, each step being defined by an index, a duration d and a parameter setting comprising the identification of the LED group(s) to be controlled and, at the start and end of the step:
the flashing frequency f of the LEDs in the LED group(s) to be controlled;
the duty cycle R of a pulse-width-modulated square-wave signal applied to the flashing frequency of the LED group(s) to be controlled; and
the luminous intensity L of the LED group(s) to be controlled, by modifying the duty cycle R of the square-wave signal.

For each LED group, the parameters can be varied in one step, with the values of f, R and L being modified and recalculated at a predetermined periodicity p.

The periodicity p is advantageously between 80 ms and 120 ms.

The frequency of the square-wave signal can be between 1 and 200 Hz.

It should be noted that the method for managing sequences of control signals from the oscillators and intended for drivers of LED groups in a brain photo-stimulation device in accordance with the invention does not have a therapeutic purpose and has no therapeutic effect for the purpose of curing or prophylaxis of a disease or organic dysfunction.

Further objects and advantages of the present invention will become apparent throughout the following description relating to embodiments which are provided only as indicative and non-limiting examples.

### BRIEF DESCRIPTION OF THE SEVERAL VIEWS OF THE DRAWINGS

The understanding of this description will be facilitated by reference to the attached drawings.

FIG. 1 shows a schematic view of a particular embodiment of the brain photo-stimulation device according to the invention, comprising a plurality of flashing light sources arranged in groups, each of the groups being managed independently of the other groups by a control means for emitting a light signal in the direction of a user.

FIG. 2 shows a second schematic view of the brain photo-stimulation device shown in FIG. 1.

### DETAILED DESCRIPTION OF THE INVENTION

With reference to the figures in the accompanying drawings, the present invention relates to a brain photo-stimulation device 1, the ultimate aim of which is to bring about a change in a user's brain activity, via a phenomenon of light flickering, or flashing, which is perceived by the user's eye.

The brain photo-stimulation device 1 conforming to the present invention is incorporated within a housing 2 advantageously in the form of a lamp that can be of various shapes and sizes.

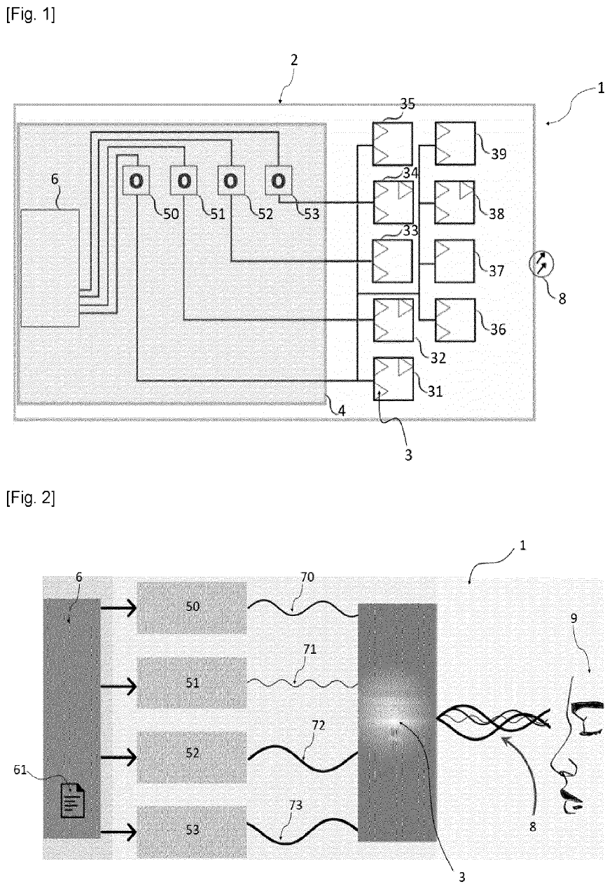

\Within this housing 2 are a plurality of flashing light sources 3, symbolized, in FIG. 1 of the attached drawings, by triangular shapes.

Advantageously, said flashing light sources 3 consist of flashing light-emitting diodes, or LEDs.

According to a preferential feature of the brain photo-stimulation device 1 of the invention, it comprises only flashing light sources, and does not incorporate any continuous light source.

It should also be noted that, advantageously, the device 1 of the invention comprises only light sources emitting white light, and no light source emitting light of any other color, which simplifies the device compared with certain existing devices in the state of the art.

In the present brain photo-stimulation device 1, as shown in the figures, the flashing light sources 3 are arranged in a plurality of groups referenced 31 to 39, each of these groups incorporating at least one flashing light source 3, consisting in particular of an LED.

Preferably, each of the groups 31 to 39 of flashing light source(s) comprises between one and three LEDs 3.

In FIG. 1, for example, the groups 31, 32, 34 and 38 each have three flashing light sources 3, while the groups 33, 35, 36 and 39 each have two flashing light sources, and the group 37 has just one.

Of course, this is only an example of a particular embodiment of the present brain photo-stimulation device 1, which should by no means be considered as limiting the invention.

In particular, the number of groups of flashing light sources, which is nine in the attached FIG. 1, can be increased or reduced, as can the number of flashing light sources 3 within each of these groups.

In addition to the plurality of flashing light sources 3 arranged in groups, the brain photo-stimulation device 1 of the invention includes means for controlling 4 the flashing frequency of said flashing light sources 3, and also the light intensity and off/on duty cycle of these light sources 3.

It should be noted that the off/on duty cycle, also commonly referred to as the “duty cycle”, corresponds, over a given period of time, to the time during which the light source is switched on with respect to the total duration of the period (time on+off).

According to a particular feature of the present brain photo-stimulation device 1, these management means 4 comprise, on the one hand, at least two oscillators and, on the other hand, means 6 for controlling said oscillators, consisting in particular of a microcontroller 6.

The microcontroller 6 drives the oscillators according to information from a computer file 61 in the form of a code for a stroboscopic light session to be projected to the user.

In a preferential, non-limiting embodiment of the invention, said brain photo-stimulation device 1 comprises more than two oscillators, and in particular three or even four oscillators, each with its own operating frequency.

It should be noted that, in a preferential embodiment, the oscillators are in the microcontroller 6.

The preferential embodiment shown in FIGS. 1 and 2 of the accompanying drawings shows a brain photo-stimulation device 1 whose means of controlling the flashing frequency, light intensity and on/off cycle of the light sources 3 comprise four oscillators 50, 51, 52 and 53.

Each of these oscillators 50, 51, 52, 53 has the function of controlling at least the flashing frequency, luminous intensity and off/on cycle of at least one group 31 to 39 comprising at least one flashing light source 3.

Thus, said oscillators 50, 51, 52, 53 control the parameters of each of the groups 31 to 39 of flashing light sources 3, from the start to the end of a session projected to a user.

In this way, each group of flashing light sources can be controlled by an oscillator independently of the other group, or groups as the case may be, of flashing light sources 3, with regard to flashing frequency, luminous intensity and off/on duty cycle.

Thus, each group 31 to 39 with at least one light source 3 generates, by means of the at least two oscillators, its own strobe frequency with its own luminous intensity and duty cycle, the addition of which with at least one other signal also having a particular strobe frequency/intensity/duty cycle, forms a light signal 8 in the direction of a user 9.

In the example shown in the attached FIG. 2, each of the four oscillators 50, 51, 52, 53 sends a command, via an electrical signal, of a strobe frequency 70, 71, 72, 73, respectively, to the group(s) of flashing light source(s) it controls.

It should be noted that, depending on the instructions transmitted by the control means 6, each of the four oscillators 50, 51, 52, 53 can control the operation of none, one or more groups of flashing light source(s) from among all the groups included in the device 1.

Simultaneous operation of the different oscillators 50, 51, 52, 53 therefore creates different variations of light parameters on separate LEDs, arranged in groups.

In short, the light flicker 8 finally emitted by the brain photo-stimulation device 1 of the invention corresponds to a combination of the different light strobe frequencies 70, 71, 72, 73 emitted by groups 31 to 39 of flashing light sources 3, under the control of oscillators 50, 51, 52, 53, controlled by the microcontroller 6.

By this means, the user 9, who is positioned in relation to the brain photo-stimulation device 1 of the invention so as to perceive, with eyes closed, the luminous flashing emitted by the flashing light sources 3 included in said device 1, will experience various effects.

On the one hand, the user perceives visual hallucinations, particularly in the form of colors, geometric patterns, movement, textures, light patterns and as a result, overall, a subjective impression of altered visual perception.

On the other hand, the user will experience an altered state of consciousness. The new brain activity, or rhythm, induced by the photo-stimulation device 1 will create a non-ordinary state of consciousness. In this non-ordinary state, certain brainwaves of higher or lower frequency, compared to the frequency of brainwaves in an ordinary state of consciousness, will appear or be more present.

It is also conceivable that the brain photo-stimulation device 1 of the invention will induce a sleep state in the user, depending on the light signals emitted.

An effect induced by means of the brain photo-stimulation device 1 of the invention may persist after the experience has ended, that is, once the light session has been completed. However, this is not the case for visual hallucinations, which are likely to develop only during the experience.

Depending in particular on the combination of light flashing frequencies controlled by the oscillators under the control of the control means 6, the brain photo-stimulation device 1 is able to lead the user 9 into a second state by a synchronization phenomenon of said user's brain electrical activity.

To this end, the control means 6, in the form of a microcontroller, act based on a computer file 61 in the form of a code of the stroboscopic light session to be projected to the user. The stroboscopic light sessions can be modified so as to adapt the light signal 8 that is ultimately emitted following the combination of light frequencies, intensities and off/on duty cycles, according to the effect on the user 9 that is sought.

Note that the control means 6 of the photo-stimulation device 1 according to the present invention can, in one embodiment, be connected to a computer application containing the digital control files of the lamp.

In this way, the user or a professional can directly select, from a smartphone or tablet in particular, a light session or sequence for the execution of the stroboscopic sequence by the photo-stimulation lamp 1.

According to a particular feature of the device 1 of the invention, said control means 6 therefore control said oscillators 50, 51, 52, 53 based on a computer file 61 in the form of a code of the stroboscopic light session to be projected to the user.

The light session in question is made up of at least two successive steps, and preferably a plurality of steps, and each of these steps is defined by an index, a duration d and, at the start and end of the step, the flashing frequency f, the off/on duty cycle R (or duty cycle of a pulse-width-modulated square-wave signal applied to the flashing frequency) and the luminous intensity L of the LEDs of the LED group(s) to be controlled, the values f, R and L being modified and recalculated at a given periodicity of between 80 ms and 120 ms.

Thus, the brain photo-stimulation device 1 of the invention enables not only precise and complex modulation of the stroboscopic light modalities, but above all optimal control of the temporal variability.

As mentioned above, modulation is performed on four main dimensions:
Frequency in Hertz
On/off duty cycle
Light intensity
Time

In other words, one of the main characteristics and the specificity of said device 1 lies in controlling the evolution of the above-mentioned parameters over time, such temporal variability enabling the device 1 to generate a light signal that is both complex and precise.

More specifically, each strobe session is composed of several steps, each defined by a duration, in seconds, and six data:
Duration (T0 to T1)
Duty Cycle at start (T0)
Light intensity at start (T0)
Frequency at start (T0)
Duty Cycle at end (T1)
Light intensity at end (T1)
Frequency at end (T1)

A stroboscopic light session can comprise hundreds of such steps, each with its own parameters, which evolve over time according to a specific algorithm.

The control means 6 included in said device 1 are designed to manage this time complexity. To ensure a smooth transition between the different “steps”, each evolution is divided into segments with a periodicity p of between 80 and 120 ms, preferably 100 ms.

This time division allows recalculation of the new value to be applied to each parameter, giving the impression of fluidity and continuity in the evolution of the stroboscopic parameters.

Example of a stroboscopic session with time variability

The example below shows a 10-second strobe session consisting of two distinct steps:

Step 1: Duration from 0 to 5 Seconds
Duty Cycle at start (T0): 20%
Light intensity at start (T0): 50%
Frequency at start (T0): 10 Hz
Duty Cycle at end (T1): 40%
Light intensity at end (T1): 70%
Frequency at end (T1): 15 Hz

### Step 2:5 to 10 Seconds

Duty Cycle at start (T0): 40%
Light intensity at start (T0): 70%
Frequency at start (T0): 15 Hz
Duty Cycle at end (T1): 60%
Light intensity at end (T1): 90%
Frequency at end (T1): 20 Hz

In this example, the time-dependent behavior is as follows:
from 0 to 5 seconds: The system starts with a duty cycle of 20%, a light intensity of 50% and a frequency of 10 Hz. During these 5 seconds, each parameter gradually evolves until T1 values are reached (40% for duty cycle, 70% for light intensity and 15 Hz for frequency).
5 to 10 seconds: The system continues to evolve from the T1 values of step 1 (which correspond to the TO values of step 2) to the T1 values of step 2.

To ensure a smooth transition, the control means 6 of the device 1, based on the computer file 61 in the form of a session code, divide each step into segments of, for example, 100 ms. Thus, during step 1, the system recalculates the values of each parameter every 100 ms to ensure a smooth transition from T0 to T1.

Thus, the present invention also relates to a method for managing sequences of control signals from oscillators 50, 51, 52, 53 to the drivers of groups 31, 32, 33, 34, 35, 36, 37, 38, 39 of light sources 3, in particular LEDs, of a brain photo-stimulation device 1 according to the invention.

In this method, at least one sequence of control signals is generated by the oscillators 50, 51, 52, 53, based on a program selected from a plurality of predefined programs stored in the control means 6.

The sequence of signals generated in this way is made up of at least two successive steps, each of which performs at least one frequency and intensity calculation for controlling the LEDs 3.

Preferentially, each successive step of the sequence is defined by an index, a duration d and a parameter setting including identification of the LED group(s) to be controlled and, at the start and end of the step:
the flashing frequency f of the LEDs in the LED group(s) to be controlled;
the off-on duty cycle R (or duty cycle of a pulse-width-modulated square-wave signal applied to the flashing frequency) of the LED group(s) to be controlled; and
the luminous intensity L of the LED group(s) to be controlled, by modifying the duty cycle R of the square-wave signal.

For each LED group, the parameters can be varied in one step, with the values of f, R and L being modified and recalculated at a predetermined periodicity p.

Thus, during this duration d of a step, the control system 6 periodically and at regular time intervals, for example according to this periodicity p which is preferentially between 80 and 120 ms, preferentially every 100 ms, checks the parameters (brightness, duty cycle, flashing frequency) applied by each of the oscillators to the LED group(s) it controls, in comparison with the information contained in the computer program that has been selected for the user.

If at least one of the parameters applied by one of the oscillators is no longer consistent with the information in the computer file, the latter is modified by the control system 6 so that the control signal sent by the oscillator to the LED group(s) it controls is once again consistent.

Conversely, if the parameters applied by each oscillator are still in line with the information in the computer file, the control system 6 makes no changes to the control signals, and carries out another check, for example 100 ms later.

Thus, a sequence or session consists more specifically of a series of instructions for a group of LEDs, controlled by an oscillator, over a given time and on certain parameters which correspond to variables inherent in stroboscopic lighting effects, namely at least the flashing frequency of the LEDs, and possibly the luminous intensity and/or an off/on duty cycle.

A sequence will give one or more groups of LEDs one or more series of modifications to this or these variable(s). In short, over a given time, each group of LEDs can follow a different setpoint, so that different stroboscopic effects accumulate to form the light signal 8 for the user 9.

In particular, a complete sequence is made up of a plurality of steps.

Each step is therefore defined by a duration and the parameter settings of the oscillators, in particular the four oscillators 50, 51, 52, 53, taking the preferential embodiment shown in FIGS. 1 and 2.

In short, each step of a lighting session is made up of a set of data linked to the evolution of the variables, such as luminous intensity, off/on duty cycle and flashing frequency, of the various oscillators 50, 51, 52, 53 for the different groups of LEDs, each step taking place over a given time.

As a reminder, each oscillator 50, 51, 52, 53 can configure several groups of LEDs 3 chosen from the groups 31 to 39 shown in FIG. 1 in the same way.

One or more oscillators 50, 51, 52, 53 can also configure a single group 31 to 39 of LEDs.

In the case of a photo-stimulation device 1 comprising four oscillators 50, 51, 52, 53, it is therefore possible to obtain a maximum of four different LED functions per step.

The oscillators 50, 51, 52, 53 can be defined in particular with the following parameters:
LED group(s) they will control;
Flashing frequency at the start and end of the step; note here that LED flashing frequency can be fixed throughout the session, that is, at the start and end of a step, and from one step to the next. This frequency can also be variable during a session, for example by increasing or decreasing linearly over a certain period of time during said session; for example, over one step of a lighting session, the flashing frequency of a group of LEDs driven by an oscillator can increase from a starting frequency of 12 Hz to an ending frequency equal to 16 Hz, over a step duration of 1 minute, for example;
Duty cycle of this flashing frequency at the start and end of the step;
LED light intensity at the start and end of the step.

Each oscillator 50, 51, 52, 53 therefore controls a specific flashing frequency for at least one group 31 to 39 of flashing light sources 3, or LEDs. The system can therefore have groups of LEDs that flash at different frequencies.

It should also be noted that several brain photo-stimulation devices 1 according to the invention can be networked so as to enable synchronized activity of these devices and the emission of different light signals 8 to the attention of a user 9.

We claim:

1. A brain photo-stimulation device, comprising:
in a housing, a plurality of flashing light sources in the form of LEDs; and
means for managing the flashing frequency of said light sources,
wherein said means for managing the flashing frequency of the light sources comprise:
at least two oscillators, each of the oscillators controlling the flashing frequency, light intensity and on/off duty cycle of at least one group comprising at least one flashing light source, and
control means for said oscillators,
wherein each group comprising at least one light source generates its own strobe frequency, which, together with at least one other strobe frequency, forms a stroboscopic light signal in the direction of a user, and
wherein said control means control said oscillators based on a computer file in the form of a code of the stroboscopic light session to be projected to the user, the session consisting of at least two successive steps, each step being defined by an index, a duration d and, at the start and end of the step, the flashing frequency, the duty cycle of a pulse-width-modulated square-wave signal applied to the flashing frequency and the luminous intensity of the LEDs of the LED group(s) to be controlled, the values being modified and recalculated at a given periodicity of between 80 ms and 120 ms.

2. The brain photo-stimulation device, according to claim 1, wherein said control means of said oscillators is comprised of a microcontroller.

3. The brain photo-stimulation device, according to claim 1, further comprising at least four oscillators, each with its own operating frequency.

4. The brain photo-stimulation device, according to claim 2, wherein the oscillators are in the microcontroller.

5. The brain photo-stimulation device, according to claim 1, further comprising at least nine groups, each comprising at least one and at most three flashing light source(s) (3), the latter consisting of LEDs.

6. A method for managing sequences of control signals from oscillators and intended for drivers of LED groups of a brain photo-stimulation device, according to claim 1, the method comprising the steps of:
generating at least one sequence of control signals by the oscillators, based on a program selected from a plurality of predefined programs stored in the control means, the sequence being comprised of at least two successive steps, each step performing a frequency and intensity calculation for LED control, each step being defined by an index, a duration d and a parameter setting comprising the identification of the LED group(s) to be controlled and, at the start and end of the step:
the flashing frequency f of the LEDs in the LED group(s) to be controlled;
the duty cycle R of a pulse-width-modulated square-wave signal applied to the flashing frequency of the LED group(s) to be controlled; and
the luminous intensity L of the LED group(s) to be controlled, by modifying the duty cycle R of the square-wave signal,
wherein, for each LED group, the parameters can be varied in one step, with the values of f, R and L being modified and recalculated at a predetermined periodicity p of between 80 and 120 ms.

7. The method of managing sequences of control signals, according to claim 6, wherein the periodicity p is equal to 100 ms.

8. The method of managing sequences of control signals, according to claim 6, wherein the frequency of the square-wave signal is from 1 to 200 Hz.
